(19) United States
(12) Patent Application Publication
(10) Pub. No.: US 2009/0311194 A1
(43) Pub. Date: Dec. 17, 2009

(54) METHODS AND COMPOSITIONS FOR THE PRODUCTION AND USE OF MAGNETOSOMES

(75) Inventors: Xiaoping Philip Hu, Tucker, GA (US); Anthony Wing Sang Chan, Norcross, GA (US); Omar Zurkiya, Boston, MA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: Emory University, Atlanta, GA (US); Georgia Tech Research Corporation, Atlanta, GA (US)

(21) Appl. No.: 11/912,817

(22) PCT Filed: Apr. 28, 2006
(86) PCT No.: PCT/US06/16432

§ 371 Date: Aug. 27, 2009

Related U.S. Application Data
(60) Provisional application No. 60/676,261, filed on Apr. 29, 2005.

(51) Int. Cl. A61K 49/00; C12N 15/87; C12N 5/06; A01K 67/027; C12N 15/00; A61K 31/7088; C12N 13/00

(52) U.S. Cl. 424/9.321; 435/440; 435/455; 435/325; 800/13; 800/14; 800/18; 800/15; 800/16; 800/17; 435/320.1; 435/173.9; 514/44 R

(57) ABSTRACT

Methods and compositions for using magnetosomes as cellular contrast agents and markers for magnetic resonance imaging are provided. Certain methods involve synthesizing magnetosomes in a cell as directed by a nucleotide construct comprising an exogenous polynucleotide sequence, wherein the magnetosome serves as a contrast agent or marker for magnetic resonance imaging. Methods of synthesizing and isolating magnetosomes for introduction into immune-matched cells within a tissue or subject for use as a contrast agent or marker for magnetic resonance imaging are also provided. Also provided are methods for stably transfecting cells to express a polypeptide that drives or modulates magnetosome production in the cell, cells produced by such methods and methods for their isolation, transgenic animals comprising at least one eukaryotic cell produced by such methods, and vectors and delivery systems for the transfection of such cells. Further provided are methods for non-invasively generating a visible image of a tissue or subject containing at least one cell such transfected cell, as well as use of such methods to monitor the location, migration, or proliferation of cells in a tissue or subject or to detect or monitor gene expression in a tissue or subject.

### FIELD OF THE INVENTION

The invention relates to the fields of molecular biology and medical imaging, more particularly to the genetic manipulation of cells to produce magnetosomes that serve as intracellular contrast agents and markers for magnetic resonance imaging and other purposes.

### BACKGROUND OF THE INVENTION

Currently, magnetic resonance imaging (MRI) is most often used to construct an image based on the intrinsic contrast provided from the relaxation of spin of hydrogen atoms. These images provide an accurate anatomical picture that has greatly advanced health care today. Clinically, the images produced by MRI are invaluable, but the full potential of using MRI in acquiring functional, physiological, and molecular information is only beginning to be realized.

Contrast agents are important in achieving such goals. Superparamagnetic iron oxide nanoparticles are regularly used for applications such as high-density magnetic storage, catalytic and separation processes (Dyal et al. (2003) J. Am. Chem. Soc., 125(7):1684-1685), and they are now playing an expanding role in magnetic resonance imaging (Bulte and Kraitchman (2004) Biomedicine, 17:484-499), in vivo tracking of stem cells and tumor progression (Bulte et al. (2001) Nat. Biotechnol., 19(12): 1141-1147; Lewin et al. (2000) Nat. Biotechnol. 18(4):410-414), cell and DNA sorting (Dressman et al. (2003) Proc. Natl. Acad. Sci. USA 100(15):8817-8822), drug delivery (Lanza et al. (2002) Acad. Radiol., 9 Suppl 2:S330-331) and cell mechanics studies (Butler and Kelly (1998) Biorheology 35(3):193-209).

Over the last decade, biocompatible particles have been linked to specific ligands for some targeted molecular imaging applications (Weissleder (1991) Magnetic Resonance in Medicine, 22:209-212; Remsen et al. (1996) American Journal of Neuroradiology, 17: 411-418; Moore et al. (1998) Biochim. Biophys. Acta. 1402(3):239-249; Bulte (1999) Proc. Natl. Acad. Sci. USA 96(26):15256-15261; Artemov et al. (2003) Magn. Reson. Med., 49(3):403-408). However, due to their relatively large size and clearance by the reticuloendothelial system (RES), there is still a lack of widespread biomedical molecular application. Imaging of macrophage activity remains the most significant application, particularly for tumor staging of the liver and lymph nodes, and several commercial products are either approved or in clinical trials. Labeling non-phagocytic cells in culture using modified particles, followed by transplantation or transfusion in living organisms, has led to an active research interest to monitor cellular biodistribution in vivo, including cell migration and trafficking.

Thus, improved contrast agents and cellular markers relating to MRI are needed for use in both research and in medical treatment, including use in the study of the dynamics of in vivo cell biology, in the detection and monitoring of disease, and in the monitoring of therapies such as those based on the use of implanted stem cells and progenitors.

### SUMMARY OF THE INVENTION

Methods for using magnetosomes as cellular contrast agents and markers for magnetic resonance imaging are provided. In certain methods, the invention relates to synthesizing magnetosomes in a cell as directed by a nucleotide construct comprising an exogenous polynucleotide sequence, wherein the magnetosome serves as a contrast agent or marker for magnetic resonance imaging or other purposes. The invention further relates to methods of synthesizing magnetosomes in a cell, isolating the magnetosomes, and introducing the magnetosomes into immune-matched cells within a tissue or subject for use as a contrast agent or marker for magnetic resonance imaging. An exemplary nucleotide sequence for use in these methods corresponds to the MagA gene (provided as SEQ ID NO: 1), but may include any nucleotide sequence encoding a polypeptide that drives or modulates magnetosome production in a target cell.

Methods for stably transfecting cells to express a polypeptide that drives or modulates magnetosome production in the cell are also provided. The invention further relates to cells produced by such methods, methods for their isolation, and transgenic animals comprising at least one eukaryotic cell produced by such methods. Such transgenic animals include, but are not limited to, mammals such as mice, rats, guinea pigs, dogs, cats, pigs, cows, goats, sheep, and non-human primates. The invention further provides vectors and delivery systems for the transfection of cells to express a polypeptide that drives or modulates magnetosome production in the cell.

Also provided are methods for non-invasively generating a visible image of a tissue or subject containing at least one cell transfected with an exogenous polynucleotide sequence to express a polypeptide that drives or modulates magnetosome production in the cell. Such imaging methods may be used to monitor the location, migration, or proliferation of such cells in a tissue or subject, including where the cell was stably transfected in vivo or was stably transfected in vitro prior to introduction of the cell into the tissue or subject. Accordingly, these methods may be used in conjunction with controllable expression techniques, for example through the use of inducible or tissue-specific promoters operably linked to the desired nucleotide sequence. These methods may also be used to detect or monitor gene expression in a tissue or subject wherein expression of a polypeptide that drives or modulates magnetosome production in a cell is coupled with expression of an additional polypeptide of interest.

### BRIEF DESCRIPTION OF THE DRAWINGS

FIG. 1 shows: a) a test vector contained MagA under control of the CMV promoter with IRES-GFP so that positively transfected cells could be identified by fluorescence; and b) a control vector contained the EGFP protein under control of the consitutively expressed CMV promoter.

FIG. 2 shows images from a Carr-Purcell-Meiboom-Gill (CPMG) sequence. a) Echo time 50 ms b) Echo time 125 ms. See Table 1 for sample descriptions numbered 1-8. At 125 ms (b), the cells transfected with MagA and supplemented with iron (sample 6) can be clearly identified on the image in comparison to other samples.

FIG. 3 shows the results of PCR confirming the presence of MagA gene in several cell lines produced by infecting 293ft cells. The 18S positive control is positive in all cell lines as expected.

FIG. 4 shows imaging data from three of the cell lines created. The images show cell pellets. Descriptions of the various samples are found in the bottom of the figure. In the bottom plot, R2 indicates the degree to which the cell line affects the MR signal. After induction, and incubation with iron, the MR signal becomes greatly affected by the formation of magnetosomes.

FIG. 5 shows electron microscopy (EM) pictures of the cell line 2B5 after it was induced with doxycycline and allowed to incubate with 200 micromolar Fe for four days. The particles can be clearly seen within the cells.

### DETAILED DESCRIPTION OF THE INVENTION

The present invention provides methods and compositions for using magnetosomes as cellular contrast agents and markers for magnetic resonance imaging. In certain methods, the invention relates to synthesizing magnetosomes in a cell as directed by a nucleotide construct comprising an exogenous polynucleotide sequence, wherein the magnetosome serves as a contrast agent or marker for magnetic resonance imaging or other purposes. The invention further relates to methods of synthesizing and isolating magnetosomes for introduction into immune-matched cells within a tissue or subject for use as a contrast agent or marker for magnetic resonance imaging. The invention further provides methods for stably transfecting cells to express a polypeptide that drives or modulates magnetosome production in the cell, cells produced by such methods and methods for their isolation, transgenic animals comprising at least one eukaryotic cell produced by such methods, and vectors and delivery systems for the transfection of such cells. The invention further provides methods for non-invasively generating a visible image of a tissue or subject containing at least one such transfected cell, as well as use of such methods to monitor the location, migration, or proliferation of cells in a tissue or subject or to detect or monitor gene expression in a tissue or subject.

Embodiments of the present disclosure employ, unless otherwise indicated, conventional techniques of synthetic organic chemistry, cell biology, cell culture, biochemistry, molecular biology, transgenic biology, microbiology, recombinant DNA, immunology, and the like, which are within the skill of the art. Such techniques are explained fully in the literature (See, e.g., Molecular Cloning, A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); the treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo (Cold Spring Harbor Laboratory Press, Cold Spring Harbor, N.Y., 1986).

In one embodiment, the invention provides a method for producing magnetosomes in a cell, the method comprising introducing into the cell a nucleotide construct comprising an exogenous polynucleotide sequence operably linked to at least one promoter that is functional in the cell, wherein the exogenous polynucleotide comprises a nucleotide sequence encoding a polypeptide that drives or modulates magnetosome production in the cell.

As used herein in the methods of the invention, including those for producing magnetosomes in a cell, the term “magnetosome” refers to any intracellular structure comprised of a magnetic mineral crystal core surrounded by a membrane, including a phospholipid membrane, regardless of the organism of origin. Magnetosomes can consist of various specific chemical compositions such as magnetite (Fe₃O₄) and greigite (Fe₃S₄) crystals, and various magnetotactic bacterial species make specific crystals or magnetosomes that have particular shapes and sizes (typically, but not exclusively, ranging from 3-120 nm long)(Bazylinski & Frankel (2004) Nature Reviews Microbiology 2:217-230). Magnetotactic bacteria orient and migrate along magnetic fields, a behavior known as magnetotaxis. Studies have shown that magnetotactic bacteria are a morphologically diverse and broad-based group of aquatic microorganisms inhabiting freshwater and marine environments ranging from aerobic to anoxic.

The magnetic crystals in magnetosomes act as small magnets within the magnetic field of the MRI imaging system causing a change in the relaxation times of the proton signal normally acquired by MRI. This change in signal causes an increase in the contrast between areas, or voxels, within the image. Cells expressing magnetic crystals may be in the form of individual cells in vitro or within an animal, or they may be a part of, or entirely comprising an individual animal. Magnetic mineral crystal (e.g., magnetosome) enhanced MRI may be then preformed on isolated magnetic crystals or cells, in vitro, or entire animals, in vivo. The shape of the magnetic mineral crystal can include shapes such as, but not limited to, rod-like, disc-like, spherical, bullet-shaped, cubooctahedral, and prismatic crystals.

As used herein in the methods of the invention, including those for producing a magnetosome in a cell, an exemplary nucleotide sequence encoding a polypeptide that drives or modulates magnetosome production in said cell corresponds to the MagA gene (provided as SEQ ID NO: 1) or variants thereof as described elsewhere herein. Other nucleotide sequences encompassed by the present invention include any nucleotide sequence encoding a polypeptide that drives or modulates magnetosome production in a target cell. Such nucleotide sequences may be derived, for example, from organisms known (or suspected) to produce magnetic crystals, including strains of magnetospirillum and related bacteria such as Magnetospirillum gryphiswaldense and Magnetospirillum magnetotacticum, as well as animals that include marine mollusks, fish, honeybees, homing pigeons, and migratory birds (See, e.g., Kirschvink (1989) Bioelectromagnetics 10:239-259; Kirschvink & Gould (1981) Biosystems 13:181-201; Wiltschko & Wiltschko (1995) Zoophysiology, 33:297; Walker et al. (1997) Nature 390:371-376; Kirschvink (1997) Nature 390:339-340; and Beason et al. (1997) Auk 114:405-415; Munro et al. (1997) Australian Journal of Zoology 45:189-198). Techniques and methods for determining whether a nucleotide sequence encodes a polypeptide that drives or modulates magnetosome production in a target cell are known to one of skill in the art (see, e.g., Nakamura et al. (1995) J. Biol. Chem., 270(47):28392-28396; Nakamura et al. (1995) J. Biochem. (Tokyo) 118(1):23-7; Bazylinski and Frankel (2004) Nat. Rev. Microbiol., 2(3):217-230; Grünberg et al. (2001) Appl Environ Microbiol., 67:4573-4582; Grünberg et al. (2004) Appl Environ Microbiol., 70:1040-1050; see also methods and techniques described in the Experimental section below).

As used herein in the methods of the invention, including those for producing a magnetosome in a cell, such methods may employ a nucleotide construct that is capable of directing the expression of at least one polypeptide, or the transcription of at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. In addition, it is recognized that methods of the present invention do not depend on the incorporation of the entire nucleotide construct into the genome, only that the cell, tissue, or subject is altered as a result of the introduction of the nucleotide construct into a cell.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

As used herein in the methods of the invention, including those for producing magnetosomes in a cell, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above.

As used herein in the methods of the invention, including those for producing a magnetosome in a cell, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. The term “exogenous DNA” or “exogenous nucleic acid sequence” or “exogenous polynucleotide” refers to a nucleic acid sequence that was introduced into a cell or organelle from an external source. Typically the introduced exogenous sequence is a recombinant sequence.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

As used herein in the methods of the invention, including those for producing a magnetosome in a cell, the term “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

The term “codon” means a specific triplet of mononucleotides in the DNA chain. Codons correspond to specific amino acids (as defined by the transfer RNAs) to start and stop of translation by the ribosome. The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

As used herein in the methods of the invention, including those for producing a magnetosome in a cell, the term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

Fragments and variants of the disclosed polynucleotides and polypeptides or proteins encoded thereby are also encompassed by the present invention. By “fragment” is intended a portion of the polynucleotide or a portion of the nucleotide sequence and hence protein encoded thereby, if any. Fragments of a nucleotide sequence may encode protein fragments that retain the biological activity of the native protein and hence drives or modulates magnetosome production in a cell. Thus, fragments of a nucleotide sequence for use in the methods of the invention will encode at least 15, 25, 30, 50, 100, 150, 200, 250, 300, 350, 400, 450, 500, 550, 600, 650, 700, 750, 800, 850, 900, 950, 1000, 1100, 1200, 1300, 1400, or 1500 contiguous amino acids, or up to the total number of amino acids present in a full-length protein or polypeptide that drives or modulates magnetosome production in a cell.

“Variant” refers to a polypeptide or polynucleotide that differs from a reference polypeptide or polynucleotide, but retains essential properties (i.e., is biologically active and hence drives or modulates magnetosome production in a cell). Exemplary variants include, for example, genes as disclosed in Grünberg et al. (1989) Bioelectromagnetics 10:239-259 that share homology with MagA in different chromosomal regions of both M. magnetotacticum and strain MC-1. As used herein, a “native” polypeptide or polynucleotide comprises a naturally occurring amino acid sequence or nucleotide sequence. For polynucleotides, conservative variants include those sequences that, because of the degeneracy of the genetic code, encode the amino acid sequence of a polypeptide that drives or modulates magnetosome production in a cell. Naturally occurring allelic variants such as these can be identified with the use of well-known molecular biology techniques. Variant polynucleotides also include synthetically derived polynucleotides, such as those generated, for example, by using site-directed mutagenesis but which still encode a polypeptide that drives or modulates magnetosome production in a cell. Generally, variants of a particular polynucleotide of for use in the methods of the invention will have at least about 40%, 45%, 50%, 55%, 60%, 65%, 70%, 75%, 80%, 85%, 87%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide for use in the methods of the invention can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the MagA protein of SEQ ID NO:2. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 87%, 90%, 91%, 92%, 93%, 94%, 95%, 96%, 97%, 98%, 99% or more sequence identity.

A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth herein.

Biologically active variants of a polypeptide for use in the methods of the invention will have at least about 40%, 45%, 50%, 55%, 60%, 65%, 70%, 75%, 80%, 85%, 87%, 90%, 91%, 92%, 93%, 94%, 95%, 96%, 97%, 98%, 99% or more sequence identity to the amino acid sequence of a protein or polypeptide that drives or modulates magnetosome production in a cell as determined by sequence alignment programs and parameters described elsewhere herein. A biologically active variant of a protein for use in the methods of the invention may differ from that protein by as few as 1-15 amino acid residues, as few as 1-10, such as 6-10, as few as 5, as few as 4, 3, 2, or even 1 amino acid residue.

By way of example, a polypeptide sequence may be identical to the reference sequence, that is be 100% identical, or it may include up to a certain integer number of amino acid alterations as compared to the reference sequence such that the percent identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given percent identity is determined by multiplying the total number of amino acids in the reference polypeptide by the numerical percent of the respective percent identity (divided by 100) and then subtracting that product from said total number of amino acids in the reference polypeptide.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Accordingly, in one embodiment of the methods of the invention for producing magnetosomes in a cell, the method comprises introducing into the cell a nucleotide construct comprising an exogenous polynucleotide sequence operably linked to at least one promoter that is functional in the cell, wherein the exogenous polynucleotide comprises: a) the nucleotide sequence set forth in SEQ ID NO:1; b) a nucleotide sequence having at least 80%, 90%, or 95% sequence identity to the sequence set forth in SEQ ID NO:1, wherein the nucleotide sequence encodes a polypeptide that drives or modulates magnetosome production in the cell; c) a nucleotide sequence comprising at least 15 contiguous nucleotides of SEQ ID NO:1 or a complement thereof; d) a nucleotide sequence encoding a polypeptide comprising the amino acid sequence set forth in SEQ ID NO:2; or e) a nucleotide sequence encoding an amino acid sequence having at least 80%, 90%, or 95% sequence identity to the sequence set forth in SEQ ID NO:2, wherein the nucleotide sequence encodes a polypeptide that drives or modulates magnetosome production in the cell. The resulting magnetosomes produced by the cells do not necessarily need to have all of the same characteristics as the native magnetosome, but are detectable by a magnetic resonance imaging system or other magnetically sensitive devices.

In another embodiment, the present invention provides a method for stably transfecting a cell to express a polypeptide that drives or modulates magnetosome production in the cell, comprising introducing into the cell a nucleotide construct comprising an exogenous polynucleotide sequence operably linked to at least one promoter that is functional in the cell, wherein the exogenous polynucleotide comprises a nucleotide sequence encoding the polypeptide. In particular embodiments, the nucleotide sequence is: a) the nucleotide sequence set forth in SEQ ID NO: 1; b) a nucleotide sequence having at least 80%, 90%, or 95% sequence identity to the sequence set forth in SEQ ID NO: 1, wherein the nucleotide sequence encodes a polypeptide that drives or modulates magnetosome production in the cell; c) a nucleotide sequence comprising at least 15 contiguous nucleotides of SEQ ID NO: 1 or a complement thereof; d) a nucleotide sequence encoding a polypeptide comprising the amino acid sequence set forth in SEQ ID NO:2; or e) a nucleotide sequence encoding an amino acid sequence having at least 80%, 90%, or 95% sequence identity to the sequence set forth in SEQ ID NO:2, wherein the nucleotide sequence encodes a polypeptide that drives or modulates magnetosome production in the cell.

In another embodiment, the invention provides a method of non-invasively generating a visible image of a tissue or subject, comprising: a) stably transfecting at least one cell located in or introduced into the tissue or subject to express a polypeptide that drives or modulates magnetosome production in the cell, the method comprising introducing into the cell a nucleotide construct comprising an exogenous polynucleotide sequence operably linked to at least one promoter that is functional in the cell, wherein the exogenous polynucleotide comprises a nucleotide sequence encoding the polypeptide; and b) scanning the tissue or subject using magnetic resonance imaging, whereby a visible image of the tissue or subject is non-invasively generated. In particular embodiments, the nucleotide sequence is: a) the nucleotide sequence set forth in SEQ ID NO: 1; b) a nucleotide sequence having at least 80%, 90%, or 95% sequence identity to the sequence set forth in SEQ ID NO: 1, wherein the nucleotide sequence encodes a polypeptide that drives or modulates magnetosome production in the cell; c) a nucleotide sequence comprising at least 15 contiguous nucleotides of SEQ ID NO: 1 or a complement thereof; d) a nucleotide sequence encoding a polypeptide comprising the amino acid sequence set forth in SEQ ID NO:2; or e) a nucleotide sequence encoding an amino acid sequence having at least 80%, 90%, or 95% sequence identity to the sequence set forth in SEQ ID NO:2, wherein the nucleotide sequence encodes a polypeptide that drives or modulates magnetosome production in the cell.

As used within these methods, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. “Transient transfection” refers to cases where exogenous nucleic acid is retained for a relatively short period of time, often when the nucleic acid does not integrate into the genome of a transfected cell, e.g., where episomal DNA is transcribed into mRNA and translated into protein. A cell has been “stably transfected” with a nucleic acid construct when the nucleic acid construct has been introduced inside the cell membrane and coding regions are capable of being inherited by daughter cells.

According to the methods of the present invention, magnetosome production in stably transfected cells can continue, even after the cells divide, since the genes have been incorporated into the cellular DNA. In this way, long-term identification of cells and cell lines can be made, even after many cell divisions. An example may be to transduce stem cells, and track their migration after injection into an animal, as described more fully below. Even after many cell divisions and migrations, cells can be located since the magnetosomes will be generated by the cells which carry the gene regardless of division or migration. Using a controllable expression system, magnetosome production can be either induced or limited as necessary. Since magnetic resonance imaging is non-invasive and the magnetosomes can be regenerated, magnetosome enhanced MRI may provide for new, longer term experiments not possible with traditional forms of contrast agents.

Accordingly, in another embodiment, the invention provides a method for monitoring the location, migration, or proliferation of cells in a tissue or subject comprising: a) stably transfecting at least one cell located in or introduced into the tissue or subject to express a polypeptide that drives or modulates magnetosome production in the cell, the method comprising introducing into the cell a nucleotide construct comprising an exogenous polynucleotide sequence operably linked to at least one promoter that is functional in the cell, wherein the exogenous polynucleotide comprises a nucleotide sequence encoding the polypeptide; and b) scanning the tissue or subject using magnetic resonance imaging, whereby a visible image of the tissue or subject is non-invasively generated and whereby the location, migration, or proliferation of cells in the issue or subject is determined. These methods encompass methods wherein the cell is located in a tissue or subject and the nucleotide construct is introduced into the cell by introducing to the tissue or subject a vector comprising the nucleotide construct, for example using tissue-specific promoters, as described more fully below, that drive expression in a cancer cell type of interest to identify and monitor cancers. Further, the methods also encompass methods wherein the nucleotide construct is introduced into the cell in vitro prior to the introduction of the cell into the tissue or subject, for example introducing the nucleotide construct into an isolated and purified stem cell in order to monitor stem cell therapy.

An analogy may be drawn between the traditional reporter gene assays routinely performed by biologists, such as assays employing β-galactosidase (β-Gal) or green fluorescent protein (GFP), and certain embodiments of the present invention. Accordingly, certain methods of the invention may be used as an alternative for other commonly used cell-screening methods. For example, a method for assessing candidate pharmaceuticals may traditionally involve contacting the candidate pharmaceutical with a cell carrying an informative reporter gene construct. Now, the standard reporter gene may be replaced with a gene encoding a polypeptide that drives or modulates magnetosome production in a cell, and the standard detection system may be replaced with an MRI system. While certain embodiments of the present invention may be used to substitute for traditional reporter gene assays, these traditional assays are far more limited in their utility. For example, traditional assays use optically-based readout technologies that are ineffective in visualizing gene expression deep within intact tissue, and often require histological processing of the biological materials. By contrast, certain embodiments of the present invention employ the use of a gene encoding a polypeptide that drives or modulates magnetosome production in a cell for use as an MRI contrast agent, allowing signal readout deep within optically opaque tissues by MRI and, if desired, readouts may be obtained with little or no disruption of the biological function of the subject material.

Numerous methods of gene delivery may be implemented within the methods of the present invention. These include, but are not limited to, chemical methods such as calcium phosphate, physical methods such as electroporation, and viral gene transfer techniques (reference: Methods in Molecular Biology: Gene Delivery to Mammalian Cells, Humana Press 2004, Volumes 245 and 246, edited by William C. Heiser). The nucleic acid may be in the form of naked DNA or RNA, associated with various proteins or the nucleic acid may be incorporated into a vector.

As used within the methods of the present invention, the term “vector” or “expression vector” is used to denote a DNA molecule, linear or circular, which includes a segment encoding a polypeptide of interest operably linked to additional segments that provide for its transcription and translation upon introduction into a host cell or host cell organelles. Such additional segments include promoter and terminator sequences, and may also include one or more origins of replication, one or more selectable markers, an enhancer, a polyadenylation signal, and the like. Expression vectors are generally derived from yeast or bacterial genomic or plasmid DNA, or viral DNA, or may contain elements of both. The term “organelle” refers to cellular membrane bound structures such as the chloroplast, mitochondrion, and nucleus and includes natural and synthetic organelles. The term “host” or “organism” or “subject” includes humans, mammals (e.g., a mouse, rat, guinea pig, dog, cat, pig, cow, goat, sheep, or non-human primate), living cells, and other living organisms. A living organism can be as simple as, for example, a single eukaryotic cell or as complex as a mammal.

Accordingly, in another embodiment, the invention provides an expression vector comprising a nucleotide construct comprising an exogenous polynucleotide sequence operably linked to at least one promoter that is functional in a target cell, wherein the exogenous polynucleotide comprises a nucleotide sequence encoding a polypeptide that drives or modulates magnetosome production in the target cell. In particular embodiments, the nucleotide sequence is: a) the nucleotide sequence set forth in SEQ ID NO: 1; b) a nucleotide sequence having at least 80%, 90%, or 95% sequence identity to the sequence set forth in SEQ ID NO:1, wherein the nucleotide sequence encodes a polypeptide that drives or modulates magnetosome production in the cell; c) a nucleotide sequence comprising at least 15 contiguous nucleotides of SEQ ID NO: 1 or a complement thereof; d) a nucleotide sequence encoding a polypeptide comprising the amino acid sequence set forth in SEQ ID NO:2; or e) a nucleotide sequence encoding an amino acid sequence having at least 80%, 90%, or 95% sequence identity to the sequence set forth in SEQ ID NO:2, wherein the nucleotide sequence encodes a polypeptide that drives or modulates magnetosome production in the cell.

Further, the expression of the exogenous polynucleotide can be controlled or placed under the control of promoters that drive expression in a constitutive, tissue-specific, or inducible manner. For example, the amount and the timing of protein expression can be regulated by using an inducible promoter. An inducible promoter is not always active the way constitutive promoters are (e.g. viral promoters). In an embodiment, the inducible promoters are activated by physical means such as the heat shock promoter. In another embodiment, the inducible promoters are activated by chemicals such as isopropyl-beta-D-thiogalactopyranoside (IPTG) or Tetracycline (Tet). For example, IPTG is a compound that can be used to activate a promoter. IPTG can be added to a system to activate a downstream gene or removed to inactivate the gene. Tetracycline can be used with an inducible on or off promoter and has the ability to regulate the strength of the promoter. The system can be constructed so that tetracycline induces expression, or such that tetracycline or doxycycline limits or stops expression.

In another embodiment, the invention relates to host cells as produced using the methods and expression systems described above. As mentioned above, the host cell may include a number of different exogenous polynucleotide of one or more modalities so that one or more imaging systems can be used. For example, MRI and positron imaging systems can be used to monitor magnetic mineral crystals and radioactive complexes.

In another embodiment, a cell line or transgenic animal can include a host cell having exogenous polynucleotide incorporated therein. In this regard, transgenic animals comprise exogenous DNA incorporated into the animal's cells to effect a permanent or transient genetic change, preferably a permanent genetic change. Permanent genetic change is generally achieved by introduction of the DNA into the genome of the cell. Vectors for stable integration include plasmids, retroviruses and other animal viruses, YACS, and the like. Generally, transgenic animals are mammals, most typically mice, but may include, for example, a rat, guinea pig, dog, cat, pig, cow, goat, sheep, or non-human primate.

The exogenous nucleic acid sequence may be present as an extrachromosomal element or stably integrated in all or a portion of the animal's cells, especially in germ cells. Unless otherwise indicated, a transgenic animal comprises stable changes to the GERMLINE sequence. During the initial construction of the animal, chimeric animals (chimeras) are generated, in which only a subset of cells have the altered genome. Chimeras may then be bred to generate offspring heterozygous for the transgene. Male and female heterozygotes may then be bred to generate homozygous transgenic animals.

Transgenic animals can be produced by any suitable method known in the art, such as manipulation of embryos, embryonic stem cells, etc. Transgenic animals may be made through homologous recombination, where the endogenous locus is altered. Alternatively, a nucleic acid construct is randomly integrated into the genome. Vectors for stable integration include plasmids, retroviruses and other animal viruses, YACS, and the like.

Numerous methods for preparing transgenic animals are now known and others will likely be developed. See, e.g., U.S. Pat. Nos. 6,252,131; 6,455,757; 6,028, 245; and 5,766,879, all incorporated herein by reference. Any method that produces a transgenic animal expressing a reporter gene following complementation or reconstitution is suitable for use in the practice of the present invention. The microinjection technique is particularly useful for incorporating transgenes into the genome without the accompanying removal of other genes.

Such transgenic animals may be useful in a variety of applications, including imaging of these animals to learn about iron distribution, the study of drug delivery in animals (wherein the drug is the inducing agent for an inducible promoter), the study of specific organs (through the use of tissue specific promoters), generation of cell lines (including stem cell lines) that can be isolated and used for various therapeutic applications, use of the transgenic animals in tissue transplant studies either as donors or recipients with non-transgenic animals.

In another embodiment, the invention further relates to methods of synthesizing magnetosomes in a cell and isolating the magnetosomes. Various applications of isolated magnetosome particles have been explored and may be used in conjunction with isolated magnetosomes produced by the methods of the present invention. For example, magnetosomes have been conjugated to antibodies and used to detect and remove E. coli cells from bacterial suspensions (Nakamura et al. (1993) Anal. Chem., 65(15): 2036-2039) as well as in the detection and quantification of the immunoglobulin, IgG (Nakamura et al. (1991) Anal. Chem., 63(3):268-272). As described more fully in the Experimental section below, magnetosomes offer a number of advantages as compared to the use of synthetic iron oxide particles in various applications, including, for example, the manufacture of magnetic tapes and printing ink, magnetic targeting of pharmaceuticals, cell separation, and contrast for MRI or other magnetic resonance devices such as relaxometers.

In one such method, magnetosomes synthesized in a cell may be isolated and introduced into immune-matched cells within a tissue or subject for use as a contrast agent or marker for magnetic resonance imaging. Because magnetosomes are coated by a membrane that comes from host cells, magnetosomes produced by immune matched host cells have less potential for triggering immune responses because they do not display foreign molecules to the host animal/human in the imaging procedure. This is an advantage over simply isolating and using magnetosomes from bacterial cells as contrast agents, as the coating on these magnetosomes would contain many molecules foreign to other animals and humans in which the contrast agent may be used.

In another embodiment, the invention further relates to methods for differentiating between live and dead cells, wherein identification of a cell expressing a polypeptide that drives or modulates magnetosome production in the cell is indicative that the cell is alive. Unlike methods in which exogenous contrast is applied, methods encompassing the use of magnetosomes produced within a given cell as a contrast agent or marker can be used to differentiate between live and dead cells, as only live cells will produce magnetosomes. In methods where exogenous contrast agent is applied, cells may die and the contrast agent taken up by other tissues such as the reticuloendothelial system. In such cases, the exogenous contrast agent would still affect the MRI but is no longer specific for the intended monitoring target.

In yet another embodiment, the present invention provides a method for isolating cells from a biological sample comprising: a) selecting a cell type of interest contained in the biological sample; b) stably transfecting at least one cell contained in the biological sample to express a polypeptide that drives or modulates magnetosome production in the cell, the method comprising introducing into the cell a nucleotide construct comprising an exogenous polynucleotide sequence operably linked to at least one promoter that is functional in the cell, wherein the exogenous polynucleotide comprises a nucleotide sequence encoding the polypeptide, and further wherein the promoter is a tissue-specific promoter that drives expression in the cell type of interest; c) dissociating the biological sample to create a suspension; d) subjecting the suspension to a magnetic field gradient, wherein cells that express the polypeptide are separated from cells that do not express the polypeptide; and e) selecting the cells that express the polypeptide.

In another embodiment, the present invention provides a method for isolating an organelle from a cell comprising: a) selecting an organelle of interest contained in the cell; b) stably transfecting the cell to express a polypeptide that drives or modulates magnetosome production in the cell, the method comprising introducing into the cell a nucleotide construct comprising an exogenous polynucleotide sequence operably linked to at least one promoter that is functional in the cell, wherein the exogenous polynucleotide comprises a nucleotide sequence encoding the polypeptide, and further wherein the promoter is an organelle localization sequence that directs the magnetosome to be localized at the selected organelle; c) lysing the cell within a liquid medium to create a sample; d) subjecting the sample to a magnetic field gradient, wherein organelles to which magnetosomes have been localized are separated from organelles to which magnetosomes have not been localized; and e) selecting the organelles to which magnetosomes have been localized.

### EXPERIMENTAL

### Background

In 1975, Richard Blakemore (Blakemore (1975) Science, 190(4212):377-379), while analyzing bacteria taken from Cape Cod marshes by microscopy, observed many bacteria swimming in only one direction. The bacteria responded to the field of a magnet placed next to the plate of bacteria. These northerly swimming bacteria were given the name “magnetotactic bacteria”, a term which now refers to many different species of bacteria identified that make chains of magnetic crystals called “magnetosomes” inside their cells. These bacteria move along the lines of a magnetic field in a process called “magnetotaxis”. It was this unique property that led to their discovery and the discovery of the magnetosome. These structures are comprised of a magnetic iron oxide crystal core, surrounded by a membrane, including a phospholipid membrane. Each species of magnetotactic bacteria has a different, but specific, type and shape of magnetosome (Schuler (1999) J. Mol. Microbiol. Biotechnol. 1(1):79-86). The magnetosomes made by magnetotactic bacteria are precise and uniform making them attractive for MR application.

Due to their difficulty to isolate and maintain in culture, only a small portion of magnetotactic bacteria have been studied. Although the complex biological process is not fully understood, there are numerous practical applications of magnetosomes in research and industry. Magnetosomes could potentially replace synthetic iron oxide particles in the manufacture of magnetic tapes and printing ink, magnetic targeting of pharmaceuticals, cell separation, and contrast for MRI (Schuler and Frankel (1999) Appl. Microbiol. Biotechnol., 52(4):464-73). Synthetic particles are often non-uniform, non-homogeneous in composition, and difficult to disperse evenly in solution. Magnetosomes, on the other hand, are highly uniform and water soluble, and produced without the need for extreme temperature, pH and pressure protocols often required for industrial synthesis of iron oxide nanoparticles.

Iron oxide nanoparticles, produced by many various synthetic means, all alter the MR signal in a similar manner. In much the same way synthetic iron oxide nanoparticles are employed, magnetosomes produced by magnetotactic bacteria can be used for MRI. This would require growing magnetotactic bacteria in large quantities, isolating the magnetosomes and subsequently using them as externally supplied contrast agents. Advantages of this approach over synthetic nanoparticle production include uniformity and potential lower cost of the bacterial magnetosomes. Additionally, phospholipid membranes surrounding the particles render them water soluble, a difficult achievement for many synthetic nanoparticle processes.

Current magnetosome work continues to advance knowledge of natural magnetosome formation within the bacteria. Although the actual process is not fully understood, increased knowledge of the genetic code required to produce bacterial magnetosomes raises the possibility of using this information to allow new cell types to generate their own internal magnetosomes. In this way, cells can internally produce their own MRI contrast agent. Cells expressing magnetosomes may be in the form of individual cells, in vitro, or within an animal. They may also be a part of, or entirely comprising an individual animal. Magnetosome enhanced MRI may be then preformed on cells or entire animals, again for both in vitro or in vivo applications. This work seeks to develop a method for internally produced iron oxide nanoparticles to serve as contrast agents for MRI.

The present examples describe the utilization of magnetosomes for MRI involving transferring the production of magnetosomes to other cell types. This concept differs from the use of isolated bacterial magnetosomes for MR contrast. In those approaches, the native magnetosome bacteria are grown in culture (see, e.g., U.S. Pat. No. 6,251,365). The magnetosomes are then purified from the bacteria to create a stock. At this point, the magnetosomes are essentially magnetic nanoparticles, which may be used in much the same way as commercially existing products such as Feridex® and monocrystalline iron oxide nanoparticles (MION). The contrast agent itself must be applied to the system or animal under study.

The approach has several advantages, as described elsewhere herein. MRI may be used to directly track specific cells producing magnetosome material. Such cells can also act as markers of other activity or disease by virtue of interaction with other cell types or biological structures. The expression of the magnetic marker can be controlled or placed under the control of an expression system. In this way, production of magnetosomes can be regulated and adjusted. This can be especially useful in preventing cells from over producing magnetosomes. Additionally, the magnetosome may serve as a marker for gene expression. In much the same way green fluorescent protein (GFP) is used in optical imaging, this system may be used for MRI.

An additional advantage over externally applied contrast agents is that long term studies are possible since: 1) the magnetosome gene(s) are incorporated into cellular DNA and are therefore reproduced into progeny cells; and 2) MR imaging in non-invasive, meaning that the same animal can be imaged over a long time course.

The present methods may also be used for the purpose of modifying the magnetosome by using genetic manipulation rather than chemical modification. For instance, many molecular imaging applications envision targeting contrast agents to specific receptors or cell surface ligands (e.g., disease specific markers). Since the magnetosome membrane comes from the host membrane, it is possible to form magnetosomes in cell lines which express desired ligands or receptors on their membranes. As a result, the magnetosome will have these molecules on their surfaces, thus avoiding the need to chemically attach such molecules to the contrast agent.

Furthermore, the present method has several advantages over methods involving transgene expression of metalloproteins from the ferritin family in specific host cells (see, e.g., Genove et al. (2005) Nature Med. 11:450-454). For example, ferritin is mammalian in origin while magnetosomes are bacterial in origin. Therefore, ferritin is already present in mammalian cells and modulation of ferritin expression could disrupt homeostasis and cause upregulation or downregulation of other genes. Also, introduction of the totally foreign magnetosomes into mammalian cells provide a more specific and unique signature of its presence. In addition, magnetosome particles are not free-form like ferritin and are not as prone to degradation as proteins.

### Example 1

MagA is known to be involved in the production of magnetosomes in the species Magnetospirillum magneticum, (Nakamura et al. (1995) J. Biol. Chem., 270(47):28392-28396; Nakamura et al. (1995) J. Biochem. (Tokyo) 118(1):23-7; Bazylinski and Frankel (2004) Nat. Rev. Microbiol., 2(3):217-230). Nakamura and co-workers (Nakamura et al. (1995) J. Biol. Chem., 270(47):28392-28396 and Nakamura et al. (1995) J. Biochem. (Tokyo) 118(1):23-7) found that MagA encodes a protein with significant sequence homology to the cation-efflux proteins, KefC, a K+-translocating protein in Escherichia coli, and NapA, a putative Na+/H+ antiporter from Enterococcus hirae. The MagA protein is present in both the cytoplasmic and magnetosome membranes of M. magneticum strain AMB-1. MagA was expressed in E. coli and inverted membrane vesicles prepared from these cells were shown to transport Fe(II) in an energy-dependent manner, leading to accumulation of Fe(II) in the vesicle, which indicates that MagA functions as a H+/Fe(II) antiporter in M. magneticum strain AMB-1. Although the process of magnetosome formation is not fully understood, it is clear that MagA plays a central role.

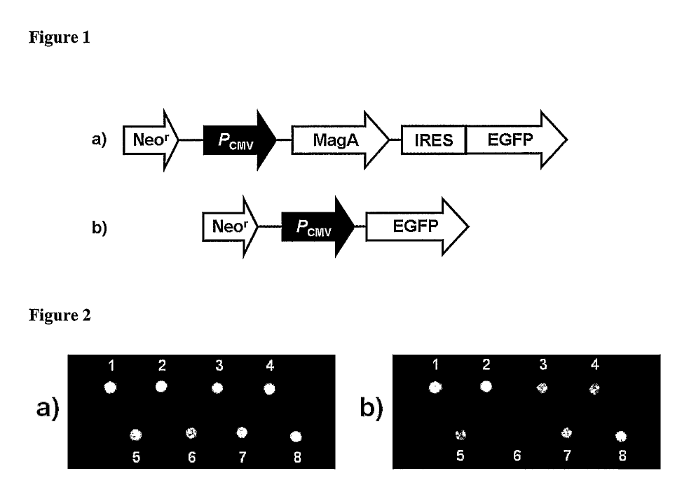

In order to test this concept, the gene was transferred to a target cell type. In this initial work, the gene was placed in a DNA expression vector under the direction of the constitutively expressed Cytomegalovirus (CMV) promoter (FIG. 1a) and transfected into 293FT cells. This vector also carried the element IRES-GFP. The IRES is an internal ribosomal entry site from which green fluorescent protein can be transcribed. In this way, fluorescence of cells was observed to confirm successful transfection.

293ft cells were grown under standard culture conditions using Dulbecco's Modified Eagle Medium (DMEM) supplemented with 10% Fetal Bovine Serum (FBS), L-Glutamine, Penicillin and Streptomycin. Calcium phosphate transfection (Heiser, ed. (2004) Methods in Molecular Biology, Vol. 1 (Humana Press, Clifton, N.J.)) was used to deliver the MagA vector as well as a control vector (FIG. 1b) expressing only GFP. Untransfected cells were also maintained as an additional control. Following transfection, media was replaced and the cells were allowed to incubate for an additional 24 hours. Transfection efficiencies of greater than 50% were observed by fluorescence microscopy. Cells were then divided into separate plates and one-half of each sample was incubated an additional 48 hours in media supplemented with 50 μM ferric citrate as a source of iron. The remaining half of each cell sample was incubated in standard media.

Cells were then trypsinized and collected. 10⁶ cells were counted from each sample and spun for 2 minutes at 1000 rpm in 15 ml falcon tubes. Cells pellets were imaged using a Siemens 3T Trio MR scanner. T2 times were calculated using a Carr-Purcell-Meiboom-Gill (CPMG) sequence. Table 1 shows the T2 results. There are eight total samples. Samples 1 and 2 show that iron supplied in the media cannot by itself alter relaxivity. This was expected since the free iron does not form a great enough magnetic moment in comparison with the magnetic domains formed by crystalline iron-oxides. Samples 3-8 contained cells and all had lower T2 times than the media only samples. Samples 3 and 4 show that cells alone did not form iron-oxide crystals, even when supplemented with iron. Sample 5 shows that cells transfected with the MagA did not form iron oxide crystals without the addition of a source of iron. Sample 6 shows that both MagA gene transfection and iron supplement did result in the formation of structures capable of altering relaxivity. Samples 7 and 8 show transfection with a control plasmid not containing MagA was not sufficient to alter relaxivity.

TABLE 1
T2 times for samples measured using
CPMG sequence on 3T Siemens TRIO.

| | | T2 (ms) |
|---|---|---|
| 1 | Media only (no Fe supplement) | 751 |
| 2 | Media only (50 μm Fe supplement) | 721 |
| 3 | 293 ft (no transfection, no Fe supplement) | 254 |
| 4 | 293 ft (no transfection, 50 μm Fe supplement) | 233 |
| 5 | 293 ft (transfected MagA, no Fe) | 259 |
| 6 | 293 ft (transfected MagA, 50 μm Fe supplement) | 106 |
| 7 | 293 ft (transfected EGFP only, no Fe supplement) | 257 |
| 8 | 293 ft (transfected EGFP only, 50 μm Fe supplement) | 237 |

FIG. 2 shows two images taken from the CPMG sequence used for T2 measurement. FIG. 2b indicates that at an echo time of 125 ms, the sample containing cells transfected with MagA and supplemented with iron had induced signal decay sufficient to differentiate it from all other samples. This indicates the feasibility of using MR imaging to identify cells prepared in the described method. Histology using iron stain (Prussian blue) with nuclear fast red counter-stain showed that the sample with MagA and iron supplement showed a blue color indicating the presence of iron.

These results indicate that a crystalline form of iron formed in the cells transfected with MagA and supplemented with iron. Free iron itself is not capable of inducing relaxivity changes by MRI as individual iron atoms do not form a large enough magnetic moment to cause dephasing of the MR signal. Histology indicates the presence of iron, and MR results indicate that some crystalline form has been produced.

### Example 2

In this example, cell lines producing magnetosomes were created. As described below, these magnetosomes could be seen by electron micrography and they affected the MR signal as expected. A major advantage of these methods is that the magnetosome production can continue, even after the cells divide, since the genes have been incorporated into the cellular DNA. In this way, long term identification of cells can be made, even after many cell divisions. Long-term studies can be conducted since the magnetosomes can be regenerated and MR imaging itself is non-invasive and does not destroy the sample being imaged. Furthermore, by developing a controllable system, long-term studies can be conducted in which cells are allowed to grow, migrate, divide and develop over a period of time in the absence of magnetosomes. This minimizes the chances of the magnetosomes to interfere with normal cellular processes. Expression of magnetosomes can then be induced and magnetosomes allowed to form, just prior to imaging protocols.

Vectors were created using an inducible promoter to allow for specific control of MagA expression using the inducible system (Tet-On™) from BD Biosciences Clontech (BD Tet-Off and Tet-On Gene Expression Systems User Manual, (2003) BD Biosciences Clontech, 55) in which expression of a gene of interest is placed under control of a “reverse” Tet repressor. The first critical component of the Tet-On system is the regulatory protein, “reverse” Tet repressor (rTetR). The resulting protein, rtTA (reverse tetracycline-controlled transactivator), is encoded by the pTet-On regulator plasmid. The second critical component is the response plasmid, which expresses a gene of interest (MagA in this case) under control of the tetracycline-response element, or TRE. The TRE is located just upstream of the minimal CMV promoter (P_{minCMV}). P_{minCMV} lacks the strong enhancer elements normally associated with the CMV immediate early promoter. Because these enhancer elements are missing, there is low background expression of the gene of interest from the TRE in the absence of binding by the rTetR domain of rtTA. The ultimate goal was to create a system containing both the regulatory and response elements so that MagA was only expressed upon binding of the rtTA protein to the TRE. For this Tet-On System, rtTA binds the TRE and activates transcription in the presence of doxycycline (Dox). Transcription is turned on or off in response to Dox in a dose-dependent manner. Although not utilized in the present example, additional potential systems include a similar Tet-Off® system in which the gene of interest is expressed upon removal of tetracycline or Dox.

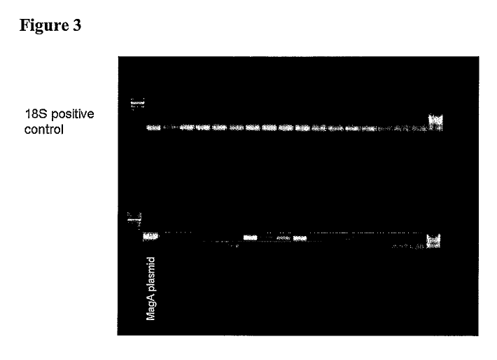

A viral vector containing the MagA gene and control elements necessary for tetracycline induction was transfected, along with standard viral elements, into a packaging cell line for production of replication defective lentivirus. This virus was used to infect 293ft cells. After several cell passages to ensure stable integration, single cells were picked and expanded to create clonal cell lines. PCR was performed on these cell lines using primers located within the MagA vector to identify positive cell lines. PCR studies confirmed the presence of MagA gene in several cell lines produced by transfecting 293ft cells (FIG. 3). The 18S positive control was positive for all cell lines, as expected.

For imaging, four plates were prepared for each cell line: 1) no doxycycline, no iron supplement, 2) no doxycycline, but with iron supplement, 3) with doxycycline, without iron, and 4) with doxycycline, with iron. In this example, the appropriate cell samples were incubated with 2 micrograms/mL doxycycline and/or 200 micromolar iron (from ferric citrate) for four days. CPMG imaging sequences were used to measure relaxation as described in Example 1.

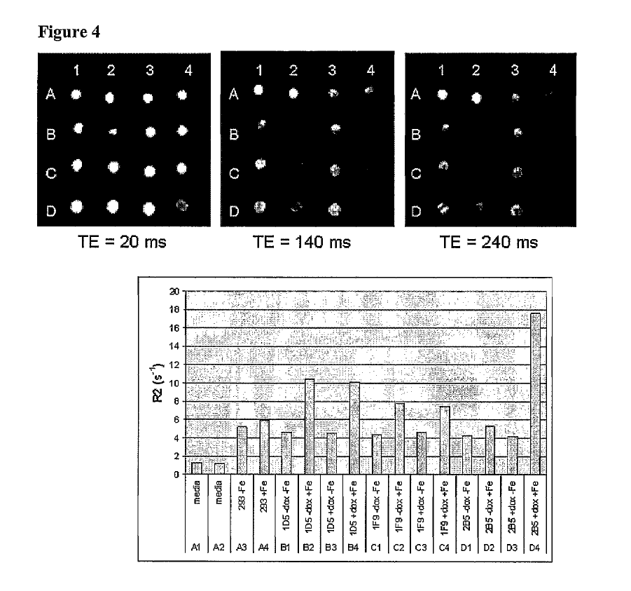

Imaging data from three of the cell lines created is provided in FIG. 4. The images show cell pellets. Descriptions of the various samples are found in the bottom of the figure. In the bottom plot, R2 indicates the degree to which the cell line affects the MR signal. Cell line 1D5 has an effect on the signal with or without induction with doxycycline. Cell line 1F9 also affects the signal with or without induction, but to a lesser extent. Cell line 2B5 has no effect on the MR signal without induction. After induction, and incubation with iron, the MR signal becomes greatly affected by the formation of magnetosomes.

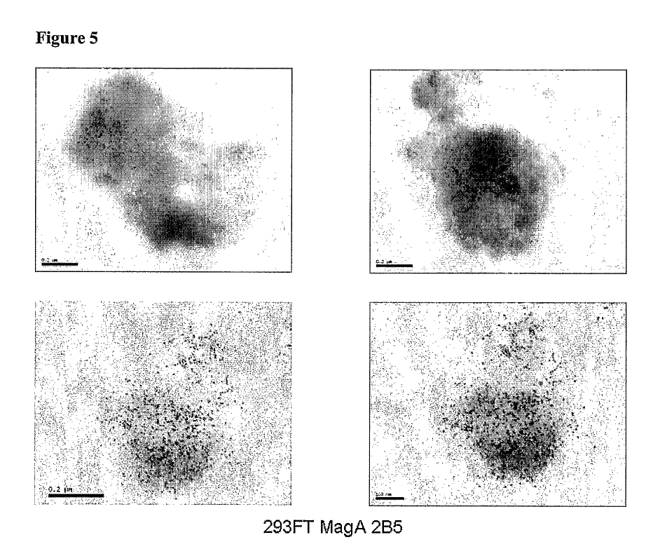

EM pictures of the cell line 2B5 after it was been induced with doxycycline and allowed to incubate with 200 micromolar Fe for four days are shown in FIG. 5. The particles can be clearly seen within the cells.

Factors involved in refining these methods in continuing work include optimization of viral transfer vectors and of lab procedures. For instance, it is not known which redox forms of iron are transported into the magnetosome vesicle in most magnetotactic bacteria, but there is evidence that Fe(II) is transported into vesicles of M. magneticum strain AMB-1 (Nakamura et al. (1995) J. Biol. Chem., 270(47):28392-28396). The uptake into vesicles, however, is a separate process from cellular uptake of iron from media and may be dependent on the cell type hosting the MagA gene. Tissue culture studies of human cells (Conrad et al. (2000) Am. J. Physiol. Gastrointest. Liver Physiol., 279(4):G767-74) show direct transport of ferrous iron, Fe(II), which is soluble at physiological pH but rapidly oxidized to ferric iron in an aerobic environment. A reducing agent, often ascorbic acid, must therefore be added to maintain the ferrous valence transiently. For the magnetotactic bacterium, M. magneticum AMB-1, it was found that ferrous sulfate and ferric gallate as iron sources enhanced magnetosome yield (Matsunaga and Tanaka (2004) Journal of Materials Chemistry, 14:2099-2105) as compared with ferric quinate, an iron chelate often used. Optimization of conditions for producing magnetosomes may be especially important for cell tracking in animals where magnetosome-producing cells may be in small numbers.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and protected by the following claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for producing a magnetosome in a cell, said method comprising introducing into said cell a nucleotide construct comprising an exogenous polynucleotide sequence operably linked to at least one promoter that is functional in said cell, wherein said exogenous polynucleotide comprises a nucleotide sequence encoding a polypeptide that drives or modulates magnetosome production in said cell.

2. The method of claim 1, wherein said nucleotide sequence is selected from the group consisting of:
a) the nucleotide sequence set forth in SEQ ID NO: 1;
b) a nucleotide sequence having at least 80% sequence identity to the sequence set forth in SEQ ID NO: 1, wherein said nucleotide sequence encodes a polypeptide that drives or modulates magnetosome production in said cell;
c) a nucleotide sequence having at least 90% sequence identity to the sequence set forth in SEQ ID NO: 1, wherein said nucleotide sequence encodes a polypeptide that drives or modulates magnetosome production in said cell;
d) a nucleotide sequence having at least 95% sequence identity to the sequence set forth in SEQ ID NO: 1, wherein said nucleotide sequence encodes a polypeptide that drives or modulates magnetosome production in said cell;
e) a nucleotide sequence comprising at least 15 contiguous nucleotides of SEQ ID NO: 1 or a complement thereof;
f) a nucleotide sequence encoding a polypeptide comprising the amino acid sequence set forth in SEQ ID NO:2;
g) a nucleotide sequence encoding an amino acid sequence having at least 80% sequence identity to the sequence set forth in SEQ ID NO:2, wherein said nucleotide sequence encodes a polypeptide that drives or modulates magnetosome production in said cell;
h) a nucleotide sequence encoding an amino acid sequence having at least 90% sequence identity to the sequence set forth in SEQ ID NO:2, wherein said nucleotide sequence encodes a polypeptide that drives or modulates magnetosome production in said cell; and
i) a nucleotide sequence encoding an amino acid sequence having at least 95% sequence identity to the sequence set forth in SEQ ID NO:2, wherein said nucleotide sequence encodes a polypeptide that drives or modulates magnetosome production in said cell.

3. The method of claim 2, wherein said nucleotide construct further comprises a nucleotide sequence encoding an additional polypeptide of interest that is also expressed in said cell.

4. The method of any of claim 2, wherein said nucleotide construct is stably incorporated into the genome of said cell.

5. The method of any of claim 2, wherein said promoter is a constitutive promoter.

6. The method of any of claim 2, wherein said promoter is a tissue-specific promoter.

7. The method of any of claim 2, wherein said promoter is an inducible promoter.

8. The method of any of claim 2, wherein said cell is isolated from a subject prior to production of said magnetosome in said cell.

9. The method of claim 8, wherein said magnetosome is isolated from said cell, further wherein said magnetosome is introduced into at least one cell in said subject in vivo for use as a contrast agent for magnetic resonance imaging.

10. A method for stably transfecting a cell to express a polypeptide that drives or modulates magnetosome production in said cell, said method comprising introducing into said cell a nucleotide construct comprising an exogenous polynucleotide sequence operably linked to at least one promoter that is functional in said cell, wherein said exogenous polynucleotide comprises a nucleotide sequence encoding said polypeptide.

11. The method of claim 10, wherein said nucleotide sequence is selected from the group consisting of:
a) the nucleotide sequence set forth in SEQ ID NO: 1;
b) a nucleotide sequence having at least 80% sequence identity to the sequence set forth in SEQ ID NO: 1, wherein said nucleotide sequence encodes a polypeptide that drives or modulates magnetosome production in said cell;
c) a nucleotide sequence having at least 90% sequence identity to the sequence set forth in SEQ ID NO: 1, wherein said nucleotide sequence encodes a polypeptide that drives or modulates magnetosome production in said cell;
d) a nucleotide sequence having at least 95% sequence identity to the sequence set forth in SEQ ID NO: 1, wherein said nucleotide sequence encodes a polypeptide that drives or modulates magnetosome production in said cell;
e) a nucleotide sequence comprising at least 15 contiguous nucleotides of SEQ ID NO: 1 or a complement thereof;
f) a nucleotide sequence encoding a polypeptide comprising the amino acid sequence set forth in SEQ ID NO:2;
g) a nucleotide sequence encoding an amino acid sequence having at least 80% sequence identity to the sequence set forth in SEQ ID NO:2, wherein said nucleotide sequence encodes a polypeptide that drives or modulates magnetosome production in said cell;
h) a nucleotide sequence encoding an amino acid sequence having at least 90% sequence identity to the sequence set forth in SEQ ID NO:2, wherein said nucleotide sequence encodes a polypeptide that drives or modulates magnetosome production in said cell; and
i) a nucleotide sequence encoding an amino acid sequence having at least 95% sequence identity to the sequence set forth in SEQ ID NO:2, wherein said nucleotide sequence encodes a polypeptide that drives or modulates magnetosome production in said cell.

12. The method of claim 11, wherein said nucleotide construct further comprises a nucleotide sequence encoding an additional polypeptide of interest that is also expressed in said cell.

13. The method of any of claim 11, wherein said promoter is a constitutive promoter.

14. The method of any of claim 11, wherein said promoter is a tissue-specific promoter.

15. The method of claims any of claim 11, wherein said promoter is an inducible promoter.

16. A cell produced by the method of any of claim 11.

17. The cell of claim 16, wherein said cell is a eukaryotic cell.

18. A transgenic animal comprising at least one eukaryotic cell of claim 17.

19. The transgenic animal of claim 18, wherein said animal is a mammal.

20. The transgenic animal of claim 19, wherein said mammal is a mouse, rat, guinea pig, dog, cat, pig, cow, goat, sheep, or non-human primate.

21. An expression vector comprising a nucleotide construct comprising an exogenous polynucleotide sequence operably linked to at least one promoter that is functional in a target cell, wherein said exogenous polynucleotide comprises a nucleotide sequence encoding a polypeptide that drives or modulates magnetosome production in said target cell.

22. The expression vector of claim 20, wherein said nucleotide sequence is selected from the group consisting of:
a) the nucleotide sequence set forth in SEQ ID NO: 1;
b) a nucleotide sequence having at least 80% sequence identity to the sequence set forth in SEQ ID NO: 1, wherein said nucleotide sequence encodes a polypeptide that drives or modulates magnetosome production in said cell;
c) a nucleotide sequence having at least 90% sequence identity to the sequence set forth in SEQ ID NO: 1, wherein said nucleotide sequence encodes a polypeptide that drives or modulates magnetosome production in said cell;
d) a nucleotide sequence having at least 95% sequence identity to the sequence set forth in SEQ ID NO: 1, wherein said nucleotide sequence encodes a polypeptide that drives or modulates magnetosome production in said cell;
e) a nucleotide sequence comprising at least 15 contiguous nucleotides of SEQ ID NO:1 or a complement thereof;
f) a nucleotide sequence encoding a polypeptide comprising the amino acid sequence set forth in SEQ ID NO:2;
g) a nucleotide sequence encoding an amino acid sequence having at least 80% sequence identity to the sequence set forth in SEQ ID NO:2, wherein said nucleotide sequence encodes a polypeptide that drives or modulates magnetosome production in said cell;
h) a nucleotide sequence encoding an amino acid sequence having at least 90% sequence identity to the sequence set forth in SEQ ID NO:2, wherein said nucleotide sequence encodes a polypeptide that drives or modulates magnetosome production in said cell; and
i) a nucleotide sequence encoding an amino acid sequence having at least 95% sequence identity to the sequence set forth in SEQ ID NO:2, wherein said nucleotide sequence encodes a polypeptide that drives or modulates magnetosome production in said cell.

23. The expression vector of claim 22, wherein said nucleotide construct further comprises a nucleotide sequence encoding an additional polypeptide of interest that is also expressed in said cell.

24. The expression vector of any of claim 22, wherein said promoter is a constitutive promoter.

25. The expression vector of any of claim 22, wherein said promoter is a tissue-specific promoter.

26. The expression vector of any of claim 22, wherein said promoter is an inducible promoter.

27. A method of non-invasively generating a visible image of a tissue or subject, said method comprising:
a) stably transfecting at least one cell located in or introduced into said tissue or subject to express a polypeptide that drives or modulates magnetosome production in said cell, said method comprising introducing into said cell a nucleotide construct comprising an exogenous polynucleotide sequence operably linked to at least one promoter that is functional in said cell, wherein said exogenous polynucleotide comprises a nucleotide sequence encoding said polypeptide; and
b) scanning said tissue or subject using magnetic resonance imaging, whereby a visible image of said tissue or subject is non-invasively generated.

28. The method of claim 27, wherein said nucleotide sequence is selected from the group consisting of:
a) the nucleotide sequence set forth in SEQ ID NO: 1;
b) a nucleotide sequence having at least 80% sequence identity to the sequence set forth in SEQ ID NO: 1, wherein said nucleotide sequence encodes a polypeptide that drives or modulates magnetosome production in said cell;
c) a nucleotide sequence having at least 90% sequence identity to the sequence set forth in SEQ ID NO: 1, wherein said nucleotide sequence encodes a polypeptide that drives or modulates magnetosome production in said cell;
d) a nucleotide sequence having at least 95% sequence identity to the sequence set forth in SEQ ID NO: 1, wherein said nucleotide sequence encodes a polypeptide that drives or modulates magnetosome production in said cell;
e) a nucleotide sequence comprising at least 15 contiguous nucleotides of SEQ ID NO: 1 or a complement thereof;
f) a nucleotide sequence encoding a polypeptide comprising the amino acid sequence set forth in SEQ ID NO:2;
g) a nucleotide sequence encoding an amino acid sequence having at least 80% sequence identity to the sequence set forth in SEQ ID NO:2, wherein said nucleotide sequence encodes a polypeptide that drives or modulates magnetosome production in said cell;
h) a nucleotide sequence encoding an amino acid sequence having at least 90% sequence identity to the sequence set forth in SEQ ID NO:2, wherein said nucleotide sequence encodes a polypeptide that drives or modulates magnetosome production in said cell; and
i) a nucleotide sequence encoding an amino acid sequence having at least 95% sequence identity to the sequence set forth in SEQ ID NO:2, wherein said nucleotide sequence encodes a polypeptide that drives or modulates magnetosome production in said cell.

29. The method of claim 27, wherein said cell is located in said tissue or subject and said nucleotide construct is introduced into said cell by introducing to said tissue or subject a vector comprising said nucleotide construct.

30. The method of claim 27, wherein said nucleotide construct is introduced into said cell in vitro prior to the introduction of said cell into said tissue or subject.

31. A method for monitoring the location, migration, or proliferation of cells in a tissue or subject, said method comprising:
a) stably transfecting at least one cell located in or introduced into said tissue or subject to express a polypeptide that drives or modulates magnetosome production in said cell, said method comprising introducing into said cell a nucleotide construct comprising an exogenous polynucleotide sequence operably linked to at least one promoter that is functional in said cell, wherein said exogenous polynucleotide comprises a nucleotide sequence encoding said polypeptide; and
b) scanning said tissue or subject using magnetic resonance imaging, whereby a visible image of said tissue or subject is non-invasively generated and whereby the location, migration, or proliferation of cells in said issue or subject is determined.

32. The method of claim 31, wherein said nucleotide sequence is selected from the group consisting of:
a) the nucleotide sequence set forth in SEQ ID NO: 1;
b) a nucleotide sequence having at least 80% sequence identity to the sequence set forth in SEQ ID NO: 1, wherein said nucleotide sequence encodes a polypeptide that drives or modulates magnetosome production in said cell;
c) a nucleotide sequence having at least 90% sequence identity to the sequence set forth in SEQ ID NO: 1, wherein said nucleotide sequence encodes a polypeptide that drives or modulates magnetosome production in said cell;
d) a nucleotide sequence having at least 95% sequence identity to the sequence set forth in SEQ ID NO: 1, wherein said nucleotide sequence encodes a polypeptide that drives or modulates magnetosome production in said cell;
e) a nucleotide sequence comprising at least 15 contiguous nucleotides of SEQ ID NO: 1 or a complement thereof;
f) a nucleotide sequence encoding a polypeptide comprising the amino acid sequence set forth in SEQ ID NO:2;
g) a nucleotide sequence encoding an amino acid sequence having at least 80% sequence identity to the sequence set forth in SEQ ID NO:2, wherein said nucleotide sequence encodes a polypeptide that drives or modulates magnetosome production in said cell;
h) a nucleotide sequence encoding an amino acid sequence having at least 90% sequence identity to the sequence set forth in SEQ ID NO:2, wherein said nucleotide sequence encodes a polypeptide that drives or modulates magnetosome production in said cell; and
i) a nucleotide sequence encoding an amino acid sequence having at least 95% sequence identity to the sequence set forth in SEQ ID NO:2, wherein said nucleotide sequence encodes a polypeptide that drives or modulates magnetosome production in said cell.

33. The method of claim 32, wherein said nucleotide construct further comprises a nucleotide sequence encoding an additional polypeptide of interest that is also expressed in said cell.

34. The method of any of claim 32, wherein said nucleotide construct is introduced into said cell in vitro prior to the introduction of said cell into said tissue or subject.

35. The method of claim 34, wherein said cell is an isolated and purified stem cell.

36. The method of any of claim 32, wherein said cell is located in said tissue or subject, and said nucleotide construct is introduced into said cell by introducing to said tissue or subject a vector comprising said nucleotide construct.

37. The method of claim 36, wherein said nucleotide construct comprises a tissue-specific promoter.

38. The method of claim 37, wherein said tissue-specific promoter drives expression in a cancer cell type of interest.

39. A method for isolating cells from a biological sample, said method comprising:
a) selecting a cell type of interest contained in said biological sample;
b) stably transfecting at least one cell contained in said biological sample to express a polypeptide that drives or modulates magnetosome production in said cell, said method comprising introducing into said cell a nucleotide construct comprising an exogenous polynucleotide sequence operably linked to at least one promoter that is functional in said cell, wherein said exogenous polynucleotide comprises a nucleotide sequence encoding said polypeptide, and further wherein said promoter is a tissue-specific promoter that drives expression in said cell type of interest;
c) dissociating said biological sample to create a suspension;
d) subjecting said suspension to a magnetic field gradient, wherein cells that express said polypeptide are separated from cells that do not express said polypeptide; and
e) selecting said cells that express said polypeptide.

40. The method of claim 39, wherein said nucleotide sequence is selected from the group consisting of:
a) the nucleotide sequence set forth in SEQ ID NO:1;
b) a nucleotide sequence having at least 80% sequence identity to the sequence set forth in SEQ ID NO: 1, wherein said nucleotide sequence encodes a polypeptide that drives or modulates magnetosome production in said cell;
c) a nucleotide sequence having at least 90% sequence identity to the sequence set forth in SEQ ID NO: 1, wherein said nucleotide sequence encodes a polypeptide that drives or modulates magnetosome production in said cell;
d) a nucleotide sequence having at least 95% sequence identity to the sequence set forth in SEQ ID NO: 1, wherein said nucleotide sequence encodes a polypeptide that drives or modulates magnetosome production in said cell;
e) a nucleotide sequence comprising at least 15 contiguous nucleotides of SEQ ID NO: 1 or a complement thereof;
f) a nucleotide sequence encoding a polypeptide comprising the amino acid sequence set forth in SEQ ID NO:2;
g) a nucleotide sequence encoding an amino acid sequence having at least 80% sequence identity to the sequence set forth in SEQ ID NO:2, wherein said nucleotide sequence encodes a polypeptide that drives or modulates magnetosome production in said cell;
h) a nucleotide sequence encoding an amino acid sequence having at least 90% sequence identity to the sequence set forth in SEQ ID NO:2, wherein said nucleotide sequence encodes a polypeptide that drives or modulates magnetosome production in said cell; and
i) a nucleotide sequence encoding an amino acid sequence having at least 95% sequence identity to the sequence set forth in SEQ ID NO:2, wherein said nucleotide sequence encodes a polypeptide that drives or modulates magnetosome production in said cell.

41. A method for isolating an organelle from a cell, said method comprising:
a) selecting an organelle of interest contained in said cell;
b) stably transfecting said cell to express a polypeptide that drives or modulates magnetosome production in said cell, said method comprising introducing into said cell a nucleotide construct comprising an exogenous polynucleotide sequence operably linked to at least one promoter that is functional in said cell, wherein said exogenous polynucleotide comprises a nucleotide sequence encoding said polypeptide, and further wherein said promoter is an organelle localization sequence that directs said magnetosome to be localized at the selected organelle;
c) lysing said cell within a liquid medium to create a sample;
d) subjecting said sample to a magnetic field gradient, wherein organelles to which magnetosomes have been localized are separated from organelles to which magnetosomes have not been localized; and
e) selecting said organelles to which magnetosomes have been localized.

42. The method of claim 41, wherein said nucleotide sequence is selected from the group consisting of:
a) the nucleotide sequence set forth in SEQ ID NO: 1;
b) a nucleotide sequence having at least 80% sequence identity to the sequence set forth in SEQ ID NO: 1, wherein said nucleotide sequence encodes a polypeptide that drives or modulates magnetosome production in said cell;
c) a nucleotide sequence having at least 90% sequence identity to the sequence set forth in SEQ ID NO: 1, wherein said nucleotide sequence encodes a polypeptide that drives or modulates magnetosome production in said cell;
d) a nucleotide sequence having at least 95% sequence identity to the sequence set forth in SEQ ID NO: 1, wherein said nucleotide sequence encodes a polypeptide that drives or modulates magnetosome production in said cell;
e) a nucleotide sequence comprising at least 15 contiguous nucleotides of SEQ ID NO: 1 or a complement thereof;
f) a nucleotide sequence encoding a polypeptide comprising the amino acid sequence set forth in SEQ ID NO:2;
g) a nucleotide sequence encoding an amino acid sequence having at least 80% sequence identity to the sequence set forth in SEQ ID NO:2, wherein said nucleotide sequence encodes a polypeptide that drives or modulates magnetosome production in said cell;
h) a nucleotide sequence encoding an amino acid sequence having at least 90% sequence identity to the sequence set forth in SEQ ID NO:2, wherein said nucleotide sequence encodes a polypeptide that drives or modulates magnetosome production in said cell; and
i) a nucleotide sequence encoding an amino acid sequence having at least 95% sequence identity to the sequence set forth in SEQ ID NO:2, wherein said nucleotide sequence encodes a polypeptide that drives or modulates magnetosome production in said cell.


SEQUENCE LISTING (2 sequences)

SEQ ID NO: 1 - DNA, 1315 bp, Magnetospirillum magneticum
  CDS (5)...(1315)
gtccatggaactgcatcatcccgaactgacctatgccgccatcgtcgccctggccgccgtgctgtgcggcgggatgatgacgcgcctgaagcagccggccgtcgtcggctacatcctggcgggggtggtgctgggacccagcggcttcgggctggtgagcaaccgcgacgccgtggccaccctggccgagttcggcgtgctgatgctgctgttcgtcatcggcatgaagctggacatcatccgttttctcgaagtgtggaagacggccatcttcaccacggttctgcagatcgccggcagcgtgggcacggccctgctgctgcgtcacggcctgggctggagcctggggctggcggtggtgctgggctgtgccgtggcggtgtcgtccaccgccgtagtgatcaaggtgctggaatcctcggacgagctggacacgccggtcggccgcaccacccttggcatcctgatcgcccaggacatggcggtggtgcccatggtgctggtgctggaatccttcgggaccaaggcgcttcttcccgccgacatggcccgggtggtgctttcggtggtgttcccggggctgctgttctggtggctgtccaagcgccgcatcgacctgccgatcaccgcccggctttcccgcgattctgaccttgccaccctgtcgaccctggcctggtgtttcggcaccgccgccatctccggcgtgctggacttgtcgccggcctatggcgccttcctgggcggcgtggtgctgggcaattccgcccagcgcgacatgctgttgaagcgtgcccagcccatcggcagcgtgctgctgatggtgttcttcctgtccatcgggctgctgctcgacttcaagttcatctggaagaatctgggcaccgttctcaccctgctggccatggtgaccctgttcaagacggcgctgaacgtcacggcgctgcgcctggcggggcaggactggcccagcgccttcctggccggcgtggccctggcccagatcggcgagttctcgttcctgctggccgagaccgacaaggcggtcaagctgatcagcgcccaggagaccaagctggtggtggcggtcaccgtgctgtccctgggtctgtcgccgttctggctgttcaccatgcggcgcatgcaccgggtggcggcggtgcatgtccattcgttccgcgatctggtcacgcggctgtatggcgacgaggcccgcgctttcgcccgcaacgcgcggcgggcccgtgtgctggtgcggcgtggttcctggagggatgaccccaatgccggacctggctctggaattagtacttag

SEQ ID NO: 2 - protein, 436 aa, Magnetospirillum magneticum
MELHHPELTYAAIVALAAVLCGGMMTRLKQPAVVGYILAGVVLGPSGFGLVSNRDAVATLAEFGVLMLLFVIGMKLDIIRFLEVWKTAIFTTVLQIAGSVGTALLLRHGLGWSLGLAVVLGCAVAVSSTAVVIKVLESSDELDTPVGRTTLGILIAQDMAVVPMVLVLESFGTKALLPADMARVVLSVVFPGLLFWWLSKRRIDLPITARLSRDSDLATLSTLAWCFGTAAISGVLDLSPAYGAFLGGVVLGNSAQRDMLLKRAQPIGSVLLMVFFLSIGLLLDFKFIWKNLGTVLTLLAMVTLFKTALNVTALRLAGQDWPSAFLAGVALAQIGEFSFLLAETDKAVKLISAQETKLVVAVTVLSLGLSPFWLFTMRRMHRVAAVHVHSFRDLVTRLYGDEARAFARNARRARVLVRRGSWRDDPNAGPGSGIST